(19) United States
(12) Patent Application Publication
(10) Pub. No.: US 2009/0081650 A1
(43) Pub. Date: Mar. 26, 2009

(54) Method for Identifying Nucleotide Sequences, Use of the Method and Test Kit

(75) Inventors: Thomas Ehben, Weisendorf (DE); Christian Zilch, Leipzig (DE)

Correspondence Address: HARNESS, DICKEY & PIERCE, P.L.C., P.O.BOX 8910, RESTON, VA, 20195, US

(73) Assignee: SIEMENS AKTIENGESELLSCHAFT, Munchen (DE)

(21) Appl. No.: 11/922,876

(22) PCT Filed: Jun. 22, 2006
(86) PCT No.: PCT/EP2006/063470

§ 371 Date: Dec. 26, 2007

(30) Foreign Application Priority Data
Jun. 27, 2005 (DE) 10 2005 029 810.9

(51) Int. Cl. C12Q 1/68

(52) U.S. Cl. 435/6

(57) ABSTRACT

A method is disclosed for identifying nucleotide sequences while using non-labeled free oligonucleotides, labeled free and hybridizable oligonucleotides and non-labeled and immobilized oligonucleotides.

### PRIORITY STATEMENT

This application is the national phase under 35 U.S.C. § 371 of PCT International Application No. PCT/EP2006/063470 which has an International filing date of Jun. 22, 2006, which designated the United States of America and which claims priority on German Patent application 10 2005 029 810.9 filed Jun. 27, 2005, the entire contents of which are hereby incorporated herein by reference.

### FIELD

At least one embodiment of the invention generally relates to a method for identifying nucleotide sequences. For example, the may relate to one which comprises carrying out the steps of reverse transcription and/or amplification, hybridization and detection, preferably in one and the same reaction chamber.

### BACKGROUND

In recent years, progress in molecular biology and the conclusion of the HUGO project, and thus the complete recording of the base sequence of human DNA, have given rise to new problems which are dealt with in a routine manner in biological basic research, in medicine and in the development of medicaments.

These problems include, for example, detection of a variation in the genome of an individual organism within the framework of genotyping. Examples of such variations are the rare point mutations in genomic DNA or the more common point mutations at a single site of the genomic DNA (Single Nucleotide Polymorphism, SNP). In the literature, SNP refers to the situation in which the particular mutation occurs in at least 1% of the organisms. Another field of application is expression analysis, i.e. determination of the degree of activity (expression) of a gene in individual cells, tissue types or organisms.

Both disciplines produce knowledge about functions, disorders and diseases or organs and tissues and are frequently applied to determining infectious diseases or in oncology, for example for tissue typing. In general, these methods can be used in all those situations in which suitable hybridizable molecules such as, for example, nucleic acids are to be determined. Thus, the field of applications comprises not only human medicine or veterinary medicine, but also forensic diagnostics, environmental and food analysis or crop protection.

Aside from the established analytical methods such as, for example, gel electrophoresis or mass spectrometry, “microarrays” have been used for this for some years now. Microarrays, sometimes also referred to as “gene chips” are the most important group of biochips.

Chemical interactions between an unknown sample substance and known reference substances take place in a microarray. Information on the unknown sample substance can be obtained by selecting suitable reference substances and observing the course and the results of said interactions. Single-stranded nucleic acid molecules which are immobilized on the inner surfaces of the reaction chambers of the microarrays (capture oligonucleotides) and which are complementary to the sample nucleic acid molecules to be detected are used as reference substances. The term ‘sample nucleic acid sequence’ refers hereinbelow also to template nucleic acid sequence or target nucleic acid sequence, and the term ‘sample nucleic acid’ also refers to template nucleic acid or target nucleic acid.

An experiment involving microarrays usually comprises the following steps:
(1) sample preparation
(2) a) amplification of the target sequence(s), where appropriate including labeling, and/or
   b) reverse transcription of the target sequence(s), where appropriate including labeling
(3) if no labeling has been carried out under (2), where appropriate additional labeling of the amplicon obtained in (2)
(4) hybridization of the unlabeled or labeled amplicons obtained in (2) and/or (3) with unlabeled capture oligo-nucleotides, and
(5) detection of the labeled hybrid molecules produced in (4).

The method will be described briefly in more detail below:
(1) Sample preparation: The sample to be analyzed (e.g. blood, saliva, tissue, plants, etc.) is prepared in a suitable manner, usually in order to concentrate its nucleic acids to be determined and to remove them from the substances interfering with the determination data.
(2a) Amplification: Usually a cell contains only a few copies of the nucleic acid sequences to be investigated which therefore must first be multiplied. This is frequently done using the polymerase chain reaction (PCR) or other methods known to the skilled worker.

In order to multiply a particular DNA sequence from a sample according to the generally familiar PCR method, the sample DNA is subjected in the presence of a DNA polymerase, the individual four deoxyribonucleotides (dATP, dGTP, dCTP and dTTP) and an oligonucleotide pair, to cycles of defined temperature fluctuations which enable the individual steps of annealing (attachment), elongation (lengthening) and denaturation to be carried out. The oligonucleotide pair is usually prepared in such a way that both oligonucleotides of said pair are composed of approx. 15 to 30 deoxyribonucleotides, their sequences being chosen in such a way that one of the two oligonucleotides is complementary to the 5′ end of the sense DNA strand of the target nucleic acid sequence (“upstream”) and the other one is complementary to the 5′ end of the antisense DNA strand (“downstream”) of the target nucleic acid sequence.

During annealing, the two oligonucleotides then hybridize in each case immediately “upstream” and “downstream” of the target sequence.

During elongation, the DNA polymerase (e.g. Taq polymerase) adds to each oligonucleotide bound to the template DNA free deoxyribonucleotides by incorporating them in 5′ to 3′ direction in accordance with the template sequence. This process initially goes beyond the end of the target sequence, resulting in single strands which end in an oligonucleotide sequence on one side and are defined by the end of the template DNA or end in a nucleotide sequence defined by the duration of the amplification step on the other side. The amplified single-stranded DNA sequences thus present in double-stranded form are referred to as amplicons.

A further denaturation then follows, and a new PCR cycle begins, i.e. separation of the DNA double strand obtained in the preceding step and annealing of oligonucleotides “upstream” and “downstream” of the target sequence. The following elongation is then already partly limited by the length of the overhang, resulting in amplicons having terminal oligonucleotides on both sides.

During the subsequent cycles, the proportion of amplicons having terminal oligonucleotides on both sides becomes prevalent over those which do not end flush with the oligonucleotide sequences on both sides.

Ideally the number of amplicons doubles with each cycle so that, for example after 30 cycles, 2³⁰ amplicons are available per sample DNA. This large number is typically sufficient for the subsequent hybridization reaction.

The PCR protocol which determines the temperatures and the particular durations of the PCR cycles mainly depends on the length and the sequence of the target sequence to be detected, the kind of polymerase used, the concentrations of additives such as, for example, Mg ions, DMSO, glycerol, etc., and the concentration of the oligonucleotides in the PCR solution.

In most cases, the target molecules (e.g. amplified DNA) are coupled with molecular markers with the aid of which the presence or the concentration of the relevant DNA molecules can be determined. Preference is given to using as markers optically active (e.g. fluorescent), magnetic, electrochemical, biological or radioactive groups which are already linked to the oligonucleotide pairs. It is also known that labeled amplicons can be obtained by incorporating previously labeled free deoxyribonucleotides during amplification.

It is possible to amplify not only one but also a plurality of target sequences from the sample during amplification. To this end, primers for these different target sequences are used in the amplification solution. In this case, the amplification reactions of these target sequences proceed simultaneously.

However, this multiplexing is frequently limited to a small number of target sequences (channels; degree of multiplexing). The limitation is mainly caused by the fact that each primer pair requires specific boundary conditions (e.g. temperature, salt content, primer concentration, composition of the amplification solution, cycle timing) for optimal amplification efficiency. The deviations of the actual amplification conditions from the particular optimal reaction conditions increase for an increasing number of primer pairs as a function of an increasing number of primer pairs in the solution.

This results in differences between the concentrations of the various PCR products (amplicons), which differences are amplified with each PCR cycle. These significant differences in concentration of the PCR products at the end of the reaction make subsequent hybridization considerably more difficult, since they result in the signal intensity during hybridization not being representative of the concentration of the corresponding target sequences to be investigated. The incorrectness of signal emission may even result in the signal intensities exceeding the dynamic measurement range of detection and making recording of individual target sequences more difficult.

(2b) Reverse Transcription (RT): The amplification step is usually dispensed with here, since in this case the mRNA which is amply produced by the cell is transcribed into cDNA in the presence of free deoxyribonucleotides and the Enzyme Reverse Transcriptase. The amount of cDNA obtained is frequently sufficient for the subsequent steps. However, where appropriate, the cDNA may also still be amplified by way of PCR (“RT-PCR”).

In this context, it has also been disclosed that labeled cDNA can be obtained by incorporating previously labeled free deoxyribonucleotides in the course of reverse transcription.

(3) Labeling: Amplified or cDNA may also be labeled in a separate step by means of a labeled probe. This probe consists of an oligonucleotide sequence provided with a marker, which is complementary to a particular section of the target sequence.

DE 198 14 001 A1, for example, describes such a method for detecting a nucleic acid by amplifying a part of the target sequence by way of two oligonucleotides in the presence of a probe with a linked reporter group and quencher group, whereby a signal can be measured after successful hybridization.

(4) Hybridization: The target molecules equipped with markers are contacted in a reaction chamber with hybridizable oligonucleotides immobilized on the inside of the reaction chamber (capture oligonucleotide). After a target molecule has hybridized with a capture oligonucleotide, markers whose signal emission is specific for a binding event accumulate at the site of its immobilization. Thus, a high signal strength suggests a high concentration of a target molecule and therefore the presence or absence, for example, of an SNP or a high degree of activation of a particular gene in the tissue.

(5) Detection: The signal may firstly be recorded inherently close to the surface. In this case, owing to the method of measurement, signals of markers which are not bound close to the substrate are not recorded, for example in the case of magnetic markers which influence a homogenous magnetic field only in immediate proximity of the substrate or in the case of fluorescent molecules in an evanescent optical field. The use of methods which do not allow inherent discrimination of the distance of the signaling markers from the substrate, for example fluorescence optics with translumination of the entire reaction chamber, usually requires washing steps prior to signal detection, which remove any markers not coupled to the base plate, in order to minimize the interfering background signal. Signal detection may also be quantitative, which is indispensable for expression analysis. The intensity of signal emission thus is a direct measure for the activity of a gene or gene section.

In typical microarrays, spots of different capture molecules may be arranged in the known manner in a pattern on one and the same support material, enabling a multiplicity of different target nucleic acid sequences (e.g. DNA or mRNA) to be determined simultaneously (in parallel). The required number of different spots increases with the increasing degree of parallelization of the studies to be carried out using an array. The sequences to be detected by different capture molecules are here amplified by way of degenerated oligonucleotide pairs or completely different oligonucleotide pairs during the PCR. This is referred to as multiplexing (see above, multiplexing). However, the number of oligonucleotide pairs need not be exactly identical to the number of spots. For example, there may be various spots having identical capture molecules, which improve the reliability of the result of the study due to their redundancy.

In order to avoid spatial inhibition of the hybridization, the capture molecules are kept at a distance from the array baseplate by way of spacer molecules. Hybridization then occurs, if a significant part of the target sequence is complementary to that of the capture sequence. In this case, marker molecules concentrate in the vicinity of the spot in question.

Microarrays are employed for different problems. In genotyping, for example, differences in individual bases on an otherwise identical DNA section (SNPS) are determined. In this case, one of the oligonucleotides must be designed in such a way that its 3′-terminal base is complementary to the base on the original or wild-type sequence. If then, in the case of an SNP of this DNA sequence, a mismatch were to occur, the 3′ base and its complementary base on the target sequence cannot form a bond, and the DNA polymerase is ultimately unable to extend the oligonucleotide. Thus no amplicons are produced that would be able to hybridize with the capture oligonucleotides, and, as a result, there will be no signal emission. Signal emission as the result of a hybridization event is thus an indicator for a perfect match of the oligonucleotide sequence with the corresponding site on the target DNA.

As an alternative to this, genotyping may be carried out by applying spots for any relevant genetic variation. The melting temperatures of the spots vary as a function of the variation present in the sample, and this can be recorded by way of signal emissions of different strengths at different temperatures of the hybridization solution.

WO 01/34842 A2 describes a method for analyzing PCR products on a biochip, which uses accordingly three types of primers: free labeled, free non-labeled, and immobilized non-labeled capture primers. The PCR produces labeled amplicons which are also extended on the capture primers.

WO 99/47701 A1 discloses a PCR method in which a single-stranded DNA molecule is mixed with a complementary primer. A second primer is complementary to the counterstrand of said DNA molecule. A third primer is immobilized and is complementary to the sequence to be amplified of said DNA molecule.

EP 1 186 669 A1 describes a PCR method which uses two free primers and one immobilized primer.

According to the prior art, the range of functions of the actual microarray, described here, is limited to the hybridization chamber required for hybridization. The steps of sample preparation (isolation of nucleic acids and, where appropriate, labeling) and amplification are carried out outside of the microarray and must be performed manually.

In some designs, the biochip includes, apart from the actual microarray, additionally microfluidic components which are used for integration of sample preparation, amplification and labeling. This is the case, for example, for the “directif®” platform from November AG. Here, a multiplicity of mechanical, fluidic and electric components must be integrated in a narrow space (“Lab-on-a-Chip”), resulting in high costs of the biochips which are usually used only once. Moreover, the function of such an integrated, miniaturized complete system is very complicated due to high complexity.

To simplify the overall process, some designs dispense with labeling of the target molecules. However, these methods are not suitable for the method of expression analysis, due to their low sensitivity.

Since all biochip platforms disclosed previously perform the individual process steps sequentially and in different reaction chambers, individual steps cannot be monitored and controlled whilst running. Thus, for example, the amplification must always be completed and cannot be terminated at a suitable earlier time.

One of the main problems of the concept of microarrays is the number of individual process steps, the required precision and the complex equipment necessary for carrying out the steps. The individual steps require special equipment, experience and knowledge and are frequently a limiting factor regarding the reproducibility of the overall result of a microarray experiment. This fact stands in the way especially of a potential transfer of the microarray concept from research to diagnostic routine.

### SUMMARY

At least one embodiment of the invention is based on establishing a method for carrying out a microarray, which reduces or avoids at least one of the disadvantages known from the prior art.

According to an embodiment of the present invention,
(I) amplification of the target molecules,
(II) hybridization, and
(III) detection are preferably carried out in one and the same reaction chamber.

This makes possible a high degree of system integration, and the process integration results in the overall process being affected to a lesser extent by fluctuations of the individual processes.

Prior to the start of the amplification, the reaction chamber of the microarray has oligonucleotides which are in each case hybridizable with the target nucleotide sequence and which are
non-labeled free, i.e. non-immobilized, oligonucleotides (abbreviated hereinbelow as “non-labeled oligo-nucleotides”),
labeled, free, i.e. non-immobilized, oligonucleotides (abbreviated hereinbelow as “labeled oligonucleotides”), and
non-labeled and immobilized oligonucleotides bound to the chamber walls of the reaction chamber or to another support material (abbreviated hereinbelow as “capture oligonucleotides”).

The above oligonucleotides, in at least one embodiment, preferably consist of from 5 to 100 and in particular from 10 to 30 or else only 15 to 25, nucleotides. The number of nucleobases is not particularly crucial for the capture oligonucleotides which therefore may alternatively also be composed of longer nucleic acid sequences.

Preferably, in at least one embodiment, the number of non-labeled oligonucleotides here exceeds the sum of labeled oligonucleotides and capture oligonucleotides. The non-labeled oligonucleotide pairs are used as starters of the subsequent primer extension to form first amplicons during the first cycles on the labeled and capture oligonucleotides.

Both sense and antisense primers for a particular target sequence are present in the solution. The ratio between non-labeled sense and antisense primers for in each case a particular target sequence can be varied over the entire range between the extreme cases “only sense” or “only antisense”. This determines the degree of asymmetric asymmetry of the PCR. The higher the differences in concentration of the non-labeled sense and antisense primers, the lower the formation of double-stranded amplicons formed free in solution. This depresses the reaction, resulting in a linear process, since the avalanche character of the reaction is less pronounced (transition of an exponential amplicon formation to a linear one).

The depression and linearization promote PCR multiplexibility of the amplification, since avalanche-like propagating concentration differences of various target sequences are not produced. A further effect of said asymmetry that promotes PCR multiplexibility of the amplification is the complete absence (in the extreme case) of unlabeled amplicons which inhibit hybridization of the labeled amplicon due to their own hybridization. A third effect of said asymmetry that promotes PCR multiplexibility of the amplification is the dependency of the course of the reaction on the elongation of labeled primers, which dependency increases as a function of the degree of asymmetry and is limited by restricting the diffusional motion of said primers caused by the mass and volume, especially of large markers, such as, for example, magnetic beads.

According to an embodiment of the invention, the primers bound to the markers (labeled oligonucleotides) are extended by means of PCR or primer extension. This results in a single strand which subsequently hybridizes with a strand complementary thereto and immobilized on a support surface, formed in another PCR reaction or primer extension, and thus acts as a functional spacer (bridge) between said marker and said support surface. As a result, the marker is immobilized according to the invention on a support material and can be detected using the abovementioned methods.

In addition, a PCR multiplex reaction can be carried out by choosing different primer pairs (sense and antisense primers). The degree of multiplexing (i.e. the number of different PCR products in a reaction) is promoted by an imbalanced concentration ratio of non-labeled sense and antisense primers in relation to corresponding labeled primers (for example oligonucleotides on a magnetic bead). For example, if all labeled sense primers are located on a voluminous marker (for example bead support) and the antisense primer is free in solution, the PCR increasingly enters the linear amplification phase after most of the bound primers have been elongated.

In this case, the concentration differences of the produced amplicons of each sequence are not as great as in a conventional PCR multiplex reaction, enabling a substantially larger number of different sequences to be detected by immobilized oligonucleotides during subsequent hybridization.

Hybridization steps can be carried out according to at least one embodiment of the invention after each PCR cycle, as soon as first hybridization events of labeled amplicons can be expected. Thus information about the dynamic hybridization behavior is obtained by comparing detection signals of successive PCR cycles with one another. As a result, amplification can be monitored, as to whether it is in an early amplification stage or in saturation, thereby achieving a substantially better quantification of the results of the measurement. Moreover, this provides a criterion for stopping the entire process. A hybridization step may then be followed again by an amplification step, where appropriate.

At least one embodiment of the invention furthermore enables large marker groups such as, for example, magnetic beads, to be used in small reaction chambers, due to the high proportion of non-labeled oligonucleotides and the comparatively lower required concentration of labeled oligonucleotides in the reaction solution.

At least one embodiment of the invention also provides the possibility of not adding any additional reagents to the reaction solution during process (I) to (III). All reagents required for the reaction can therefore either be premixed or be stored in the reaction chamber in a dry state.

In accordance with at least one embodiment of the invention, readout should take place close to the surface, since this takes full advantage of the simple fluidics due to dispensing with washing steps.

The microarrays described at the outset of the specification and the method described there may be used for the method of at least one embodiment of the invention, provided that the method still has the features of at least one embodiment of the present invention.

In an embodiment of the invention, according to which the latter is employed for expression analysis of particular genes, essentially two methods may be used.

(1) The method of the invention, as illustrated above, can be adopted by putting the step of reverse transcription before amplification (e.g. by RT-PCR).

(2) The amplification step may be dispensed with entirely, if a sufficient amount of mRNA is present in the sample, the step of amplification then being a reverse transcription which translates said mRNA into cDNA with simultaneous labeling.

At least one embodiment of the invention therefore furthermore relates to the presence of labeled oligo(T) nucleotides and to the use of reverse transcriptase. The capture oligonucleotides chosen here may have to be extended accordingly in order to achieve the desired hybridization, and therefore will typically consist of from 20 to 100, preferably from 20 to 50, nucleobases.

The method of at least one embodiment of the invention may be applied to all fields in which nucleic acid analyses are carried out, such as, for example, in medical, forensic, food and environmental analysis, in crop protection, veterinary medicine or generally in life science research.

Since the PCR initially takes place predominantly with unlabeled oligonucleotides, unlabeled amplicons are thus produced (FIG. 1). Since the concentration of non-labeled oligonucleotides decreases in the course of the PCR cycles, annealing (FIG. 3), elongation (FIG. 4) and denaturation events (FIG. 5) involving labeled oligonucleotides and capture oligonucleotides increasingly occur.

Hybridizations between elongated oligonucleotides produced from labeled oligonucleotides and from capture oligonucleotides are initiated according to the invention by maintaining a temperature above the denaturation temperature hybridization temperature, after the end of the denaturation step of a PCR cycle (FIG. 6). This prevents still free oligonucleotides from attaching tightly to the in each case complementary sites of the amplicons. In contrast, the PCR-produced capture amplicons immobilized on the reaction chamber wall or otherwise now hybridize with their complementary, labeled amplicons from the reaction solution. Since their sequence is substantially longer than that of the labeled and non-labeled oligonucleotides (for example by a factor of from 2 to 100, preferably 10 to 50), said hybridization remains stable even under the prevailing temperature which is higher than the annealing temperature. The prevailing temperature thus should be above the melting temperature of the relatively short primers and below the melting temperature of the relatively long amplicons.

### BRIEF DESCRIPTION OF THE DRAWINGS

Embodiments of the invention will be illustrated by way of example on the basis of the description of the figures below.

FIGS. 1 to 6 depict the diagrammatic course of the process. The target sequence. (S0) is bounded by the sequences S1 and S2, it being possible in general for S1 or S2 also to still be part of the target sequence. The in each case complementary sequences are indicated by “S0 overscore”, “S1 overscore” or “S2 overscore”. In the text below, these are referred to as S0*, S1* and S2*.

### DETAILED DESCRIPTION OF THE EXAMPLE EMBODIMENTS

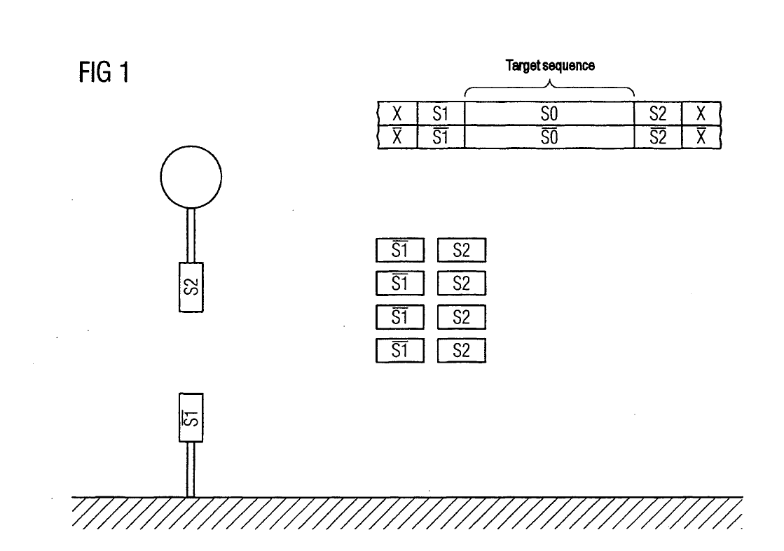

FIG. 1 depicts the start of the PCR reaction, wherein the immobilized oligonucleotides (having the sequence S1*) and labeled oligonucleotides (having the sequence S2) may be present according to an embodiment of the invention in a markedly lower concentration than the non-labeled oligonucleotides (S1* and S2). They will therefore be first to undergo amplification with the target sequence (S0) or (S0*) or (S1+S0+S2) or (S1*+S0*+S2*).

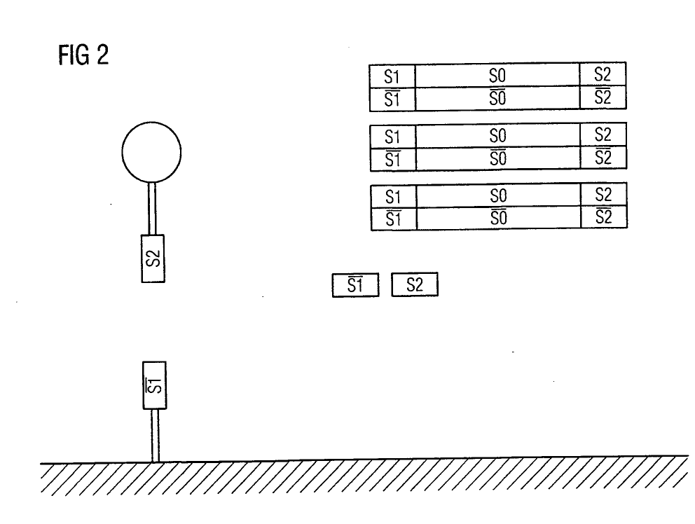

FIG. 2 depicts the course of a PCR reaction which, up to then, mainly has generated only amplicons using the majority of non-labeled oligonucleotides (S1* and S2).

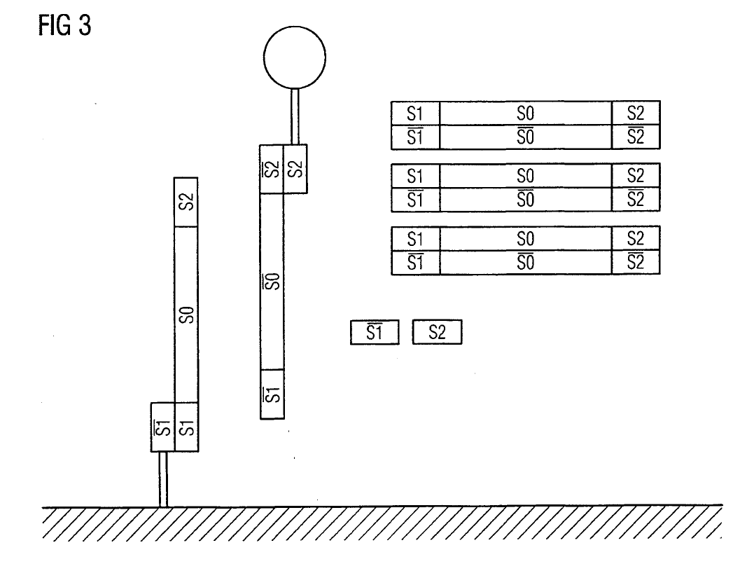

FIG. 3 depicts the further course of the process of an embodiment of the invention, with the amplicons generated previously according to FIG. 2 now serving as templates for the labeled (S2) and capture oligonucleotides (S1*).

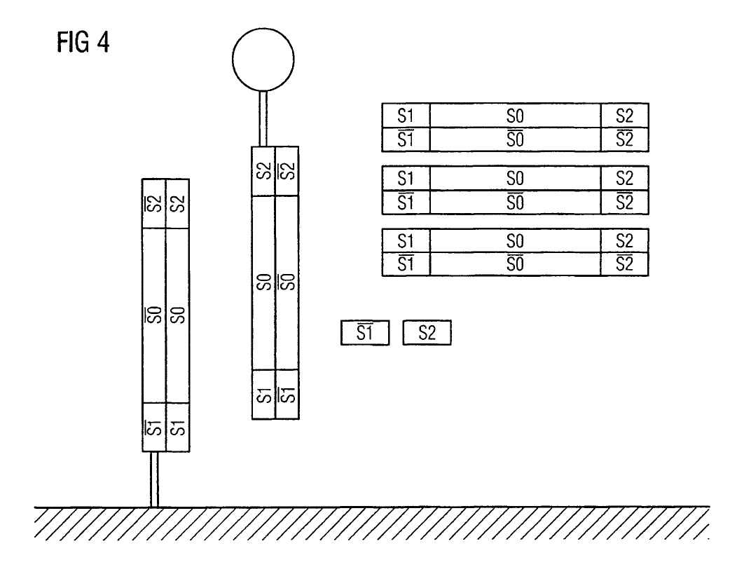

FIG. 4 depicts the fully extended amplicon of FIG. 3.

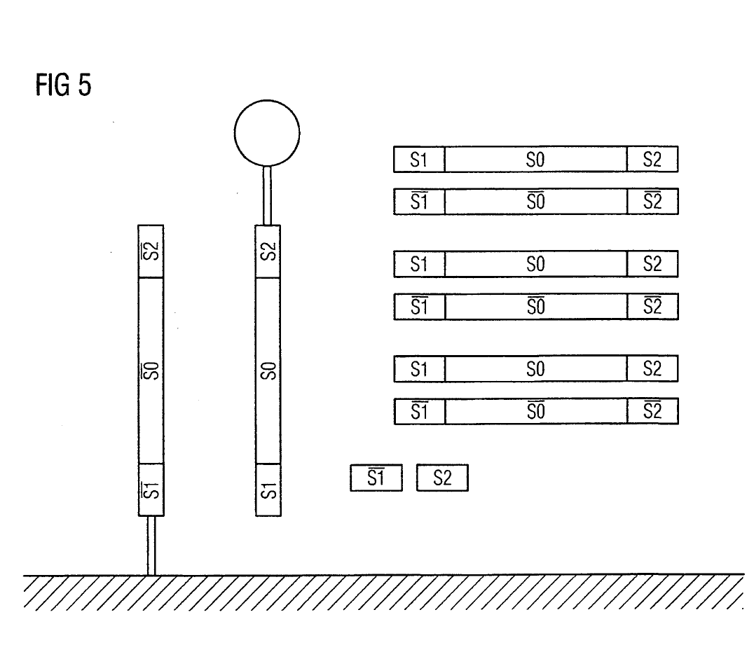

FIG. 5 depicts the double strands generated in FIG. 4 being separated to give in each case two single strands (amplicons), the separation caused by temperature increase, for example.

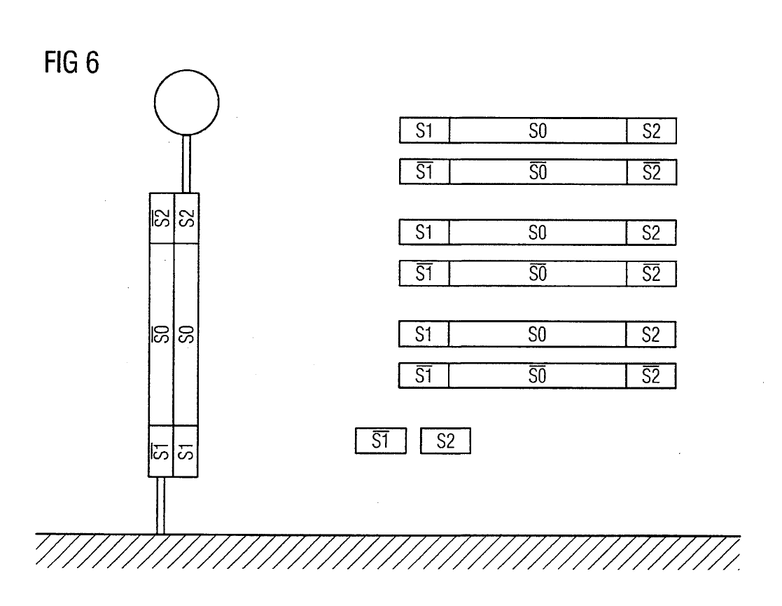

FIG. 6 finally depicts the hybridization of an embodiment of the invention between the obtained labeled and capture amplicons of FIG. 5. Although the unlabeled oligonucleotides dissolved at higher concentrations compete with the labeled oligonucleotides, this should not impede sufficient hybridization of labeled oligonucleotides, since the density of the capture amplicons produced there is so high that the concentration of amplicons decreases in the immediate vicinity of the spot, as a result of which saturation of said capture molecules of said spot is not the limiting factor during hybridization with labeled amplicons.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for identifying at least one target nucleotide sequence, comprising:
providing oligonucleotides hybridizable with at least one target nucleotide sequence, which are
non-labeled, free oligonucleotides, referred to as non-labeled oligonucleotides hereinbelow,
labeled, free, hybridizable oligonucleotides, referred to as labeled oligonucleotides hereinbelow, and
non-labeled and immobilized oligonucleotides, referred to as capture oligonucleotides hereinbelow;
at least one of
providing the sample substance to the reaction chamber, said sample substance comprising the at least one target nucleotide sequence and
preparing the at least one target nucleotide sequence from said sample substance by reverse transcription, the reverse transcription including
carrying out a reverse transcription of an RNA in the reaction chamber to prepare at least one of labeled and non-labeled target nucleotide sequence;
at least one of
amplification comprising the at least one target nucleotide sequence, the non-labeled oligonucleotides, the labeled oligonucleotides and the capture oligonucleotides to prepare labeled amplicons, non-labeled amplicons and capture amplicons, and
leaving out said amplification, if a sufficient number of target nucleotide sequences has been obtained by reverse transcription by way of the labeled oligonucleotides and, in this case, the provided oligonucleotides being available for preparing labeled nucleotide sequences;
at least one of
hybridizing the labeled amplicons with the immobilized capture amplicons, the density of the capture amplicons formed being so high that the concentration of free labeled and non-labeled amplicons decreases in the immediate vicinity of said capture amplicons, as a result of which saturation of said capture amplicons is not the limiting factor during hybridization with labeled amplicons,
hybridizing the labeled nucleotide sequences with the capture oligonucleotides; and
detecting the capture amplicons or the capture oligonucleotide,
all reagents required for carrying out the method being present in a single reaction chamber and nothing being added during the further course of the method.

2. The method as claimed in claim 1, further comprising
carrying out another amplification.

3. The method as claimed in claim 2, wherein the method steps are carried out in one and the same reaction chamber.

4. The method as claimed in claim 1, wherein the method steps are case carried out in a single reaction chamber.

5. The method as claimed in claim 1, wherein the capture oligonucleotides are immobilized to the reaction chamber.

6. The method as claimed in claim 1, wherein the labeled oligonucleotides are coupled to beads.

7. The method as claimed in claim 1, wherein amplification comprises a PCR comprising a number of denaturation, annealing and elongation cycles under the influence of a temperature cycle.

8. The method as claimed in claim 1, wherein the sequences to be investigated consist of DNA and the oligonucleotides consist of 5 to 1000.

9. The method as claimed in claim 1, wherein the markers are determined at least one of optically, electrochemically, enzymatically, magnetically, gravimetrically, radioactively or by hapten/antibody interactions, and have or generate charge carriers.

10. The method as claimed in claim 1, wherein the method comprises several sequences of the detecting and hybridizing step.

11. The method as claimed in claim 1, wherein the markers are detected close to the surface, where appropriate through the solution.

12. The process as claimed in claim 1, wherein the concentration of the non-labeled oligonucleotides is higher than the sum of the labeled oligonucleotides and the capture oligonucleotides.

13. The method as claimed in claim 1, wherein at least 2, different non-labeled oligonucleotides and at least 2 different labeled oligonucleotides hybridize withy in each case at least 2 different target sequences present in the sample substance in a multichannel multiplexing method.

14. The use of the method as claimed in claim 1 for genotyping or SNP analysis comprising amplification according to claim 1.

15. The use of the method as claimed in claim 1 for gene expression analysis, where appropriate additionally comprising amplification according to claim 1.

16. The use as claimed in claim 15, wherein the sequences to be investigated consist of RNA and the oligonucleotides consist of 5 to 1000, nucleobases.

17. A test kit having only a single reaction chamber, comprising:
non-labeled, free oligonucleotides, referred to as non-labeled oligonucleotides hereinbelow;
labeled, free oligonucleotides, referred to as labeled oligonucleotides hereinbelow; and
non-labeled and immobilized oligonucleotides, referred to as capture oligonucleotides hereinbelow;
support material for said capture oligonucleotides, present as part of the reaction chamber or located in the reaction chamber, said capture oligonucleotides being immobilized in high density to the support material; and
reagents comprising at least a reaction solution, enzymes, free deoxyribonucleotides, buffers and additives.

18. The test kit as claimed in claim 17, further comprising at least one of the following substances as additives: DMSO, glycerol, and Mg ions.

19. The test kit as claimed in claim 17, wherein the number of non-labeled oligonucleotides added is higher than the sum of the labeled oligonucleotides and the capture oligonucleotides added.

20. The test kit as claimed in claims 17, wherein at least 2 different non-labeled oligonucleotides and at least 2, different labeled oligonucleotides hybridize with in each case at least 2 different target sequences present in the sample substance in a multichannel multiplexing method.

21. A method, comprising:
using the test kit as claimed in claim 17.

22. The method as claimed in claim 8, wherein the sequences to be investigated consist of DNA and the oligonucleotides consist of 10 to 100 nucleobases.

23. The method as claimed in claim 22, wherein the sequences to be investigated consist of DNA and the oligonucleotides consist of 15 to 30 nucleobases.

24. The method as claimed in claim 1, wherein at least 5 different non-labeled oligonucleotides and at least 5 different labeled oligonucleotides hybridize with in each case at least 5 different target sequences present in the sample substance in a multichannel multiplexing method.

25. The use as claimed in claim 16, wherein the sequences to be investigated consist of RNA and the oligonucleotides consist of 10 to 100 nucleobases.

26. The use as claimed in claim 25, wherein the sequences to be investigated consist of RNA and the oligonucleotides consist of 15 to 30 nucleobases.

27. The test kit as claimed in claim 17, wherein at least 5 different non-labeled oligonucleotides and at least 5, different labeled oligonucleotides hybridize with in each case at least 5 different target sequences present in the sample substance in a multichannel multiplexing method.
